(19) United States
(12) Plant Patent Application Publication
(10) Pub. No.: US 2010/0218294 P1
(43) Pub. Date: Aug. 26, 2010

(54) Agaricus bisporus mushroom plant named J10165

(50) ;

(76) Inventors: Richard W. Kerrigan, Kittanning, PA (US); Mark P. Wach, Allison Park, PA (US)

Correspondence Address: RENNER KENNER GREIVE BOBAK TAYLOR & WEBER, FIRST NATIONAL TOWER, SUITE 400, 106 SOUTH MAIN STREET, AKRON, OH, 44308-1412, US

(21) Appl. No.: 12/380,199

(22) Filed: Feb. 25, 2009

(51) Int. Cl. A01H 15/00

(52) U.S. Cl. PLT/394

(57) ABSTRACT

A new and unique variety of the mushroom Agaricus bisporus (J. Lange) Imbach is described. It exhibits an attractive appearance that includes a smooth, bright white cap, and is biologically incompatible with strains of the ‘U1’ lineage group.

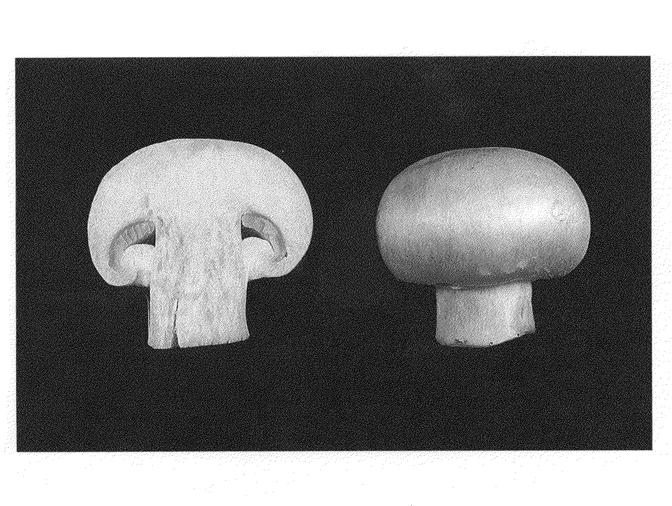

### LATIN NAME OF GENUS AND SPECIES

Agaricus bisporus (J. Lange) Imbach

### VARIETAL DENOMINATION

J10165

### BACKGROUND OF THE INVENTION

The present invention relates to a new and distinct variety of mushroom plant of Agaricus bisporus (J. Lange) Imbach. The plant of the present invention is an edible mushroom.

The edible mushroom Agaricus bisporus (J. Lange) Imbach, a basidiomycete fungus, is widely cultivated around the world. In Europe and North America, it is the most widely cultivated mushroom species. The value of the annual Agaricus bisporus mushroom crop in the United States was about $920,000,000 in 2003-2004, according to the National Agricultural Statistics Service, Agricultural Statistics Board, U.S. Department of Agriculture (Aug. 16, 2004). More than 90 percent of the Agaricus mushrooms cultivated in the United States, Europe, and elsewhere have a white pileus color, in accordance with consumer preferences.

Approximately 25 years ago, the first two white hybrid strains of A. bisporus, developed by a laboratory at Horst, the Netherlands, were introduced into commercial cultivation. These two “Horst” strains, called ‘U1’ and ‘U3,’ are closely related crosses between two pre-existing white cultivated strains, as per M. Imbernon et al., Mycologia, 88(5), 749-761 (1996), herein incorporated by reference. The ‘U1’ and ‘U3’ strains, while still cultivated at present, are additionally thought to be the direct progenitors of all other white A. bisporus mushrooms currently cultivated in most regions of the world.

Commercial mushroom strains developed from ‘U1’ and ‘U3,’ such as ‘A15’ and ‘S130,’ are all either clones of ‘U1’ or ‘U3,’ being developed by clonal vegetative propagation, or quasi-clones derived from spores which retain the great majority of the parental genotype, as shown by R. W. Kerrigan et al. in Genetics, 133, 225-236 (1993), herein incorporated by reference. A group of strains developed either by cloning or by spore propagation, or both, from a single progenitor (as opposed to outcrossing between two different progenitors) is called a lineage group. Except for minor acquired genetic differences all white strains developed within the Horst ‘U1’ lineage group and Horst ‘U3’ lineage group share a single basic genotype with the original ‘U1’ or ‘U3’ strains, respectively (which are themselves very similar, due to their close relationship). For these reasons, and the fact that the Horst ‘U3’ lineage group is presently cultivated to a much smaller extent than the Horst ‘U1’ lineage group, modern white Agaricus mushroom cultivation is effectively a monoculture.

Currently, the most commercially successful representative of the Horst ‘U1’ and Horst ‘U3’ lineage groups is a strain designated ‘A15’ owned by the assignee of record. That strain, specifically, is from the Horst ‘U1’ lineage group.

The introduction of new varieties of white Agaricus bisporus mushrooms into commercial culture has been impeded by three difficulties. First, cross-breeding strains of Agaricus bisporus var. bisporus can be difficult and cumbersome. U.S. Pat. No. 5,304,721 sets forth many of the problems associated with cross-breeding. Second, experience indicates that most wild germ plasm resources for this species exhibit various traits that would be unacceptable in the marketplace. Third, most of these germ plasm resources incorporate alleles that give rise to brown mushrooms, which are in less demand by consumers than are white mushrooms. Color is predominately determined by alleles at the Ppc-1 locus. A more detailed description of these color determinative alleles is set forth in P. Callac et al., Fungal Genetics and Biology, 23(2): 181-188 (1996), the disclosure of which is incorporated herein by reference.

Alleles providing the white color trait are rare to relatively uncommon in most wild populations of A. bisporus. Of approximately 150 wild Agaricus bisporus mushroom strains collected in coastal California, only 2 were white, while the rest were brown, as reported in R. W. Kerrigan et al., Molecular Ecology 7(1): 35-45 (1998), incorporated herein by reference.

The difficult nature of breeding a commercially successful hybrid variety of A. bisporus is illustrated by the fact that very few patent applications for novel hybrid Agaricus bisporus strains have been filed in the United States; of these, only one (i.e., assignee of record's brown hybrid strain ‘X618,’ marketed as ‘S600’) has enjoyed even moderate commercial success. It is believed that no hybrid white mushrooms other than ‘U1’ and ‘U3’ have heretofore ever been successfully introduced into commerce in the United States.

There is a wide range of potential benefits to introducing greater diversity of strains into commercial cultivation. Novel strains may exhibit novel patterns of nutritional resource utilization, different responses to environmental manipulation, precocity or different developmental schedules, and novel aesthetic and culinary properties for the consumer. Examples of traits favored by the consumer include a smooth, bright white cap surface. Some benefits may become apparent only after years of cultivation and marketing experience, for example, if a novel crop pathogen or cultivation technique emerges.

New strains may offer improved resistance to known and emerging diseases of the crop. In particular, they are very likely to be much less susceptible to infection by established viral diseases that are transmitted by anastomosis (i.e., the fusion of fungal cells, called hyphae). Empirically, it is known that, for two individual heterokaryotic strains of basidiomycete fungi, anastomosis is almost always impossible or difficult and generally unsuccessful. A more detailed description of anastomosis and of some viral diseases to which basidiomycete fungi are susceptible can be found in A. S. M. Sonnenberg et al., Mushroom Science 14, 587-594 (1995), incorporated herein by reference.

### SUMMARY OF THE INVENTION

The present invention is a new and distinct variety of Agaricus bisporus mushroom characterized by abundant production of mushrooms having smooth, white caps. The new mushroom also has a genotype that combines markers from each of its progenitors, forming a unique genotype with respect to other known white hybrid mushrooms. This novel and distinct variety of mushroom is identified as A. bisporus hybrid ‘J10165’.

The present invention is a new and distinct hybrid variety of the button mushroom A. bisporus (J. Lange) Imbach, and is named ‘J10165’. This new hybrid mushroom variety has been asexually reproduced by vegetative mycelial propagation in Kittanning, Pa., in the breeding program of Sylvan Research, 198 Nolte Dr., Kittanning, Pa. 16201.

To vegetatively propagate the mushroom culture aseptically, under laboratory conditions, a small portion of a pure (=axenic) mycelial culture on a suitable medium, such as potato dextrose agar (PDA), is transferred to a fresh plate or tube of newly prepared, sterilized medium (for example PDA) using a sterilized instrument such as a scalpel. Any aseptic transfer of an axenic culture to fresh culture medium achieves the objective of vegetative propogation. These techniques are standard and absolutely routine in the mushroom cultivation industry.

### BRIEF DESCRIPTION OF THE DRAWING

The drawing illustrates the appearance of a mushroom, sliced in cross-section, produced by the A. bisporus hybrid ‘J10165’ culture grown on a substrate of composted straw and harvested and trimmed per commercial practice. The mushroom shown is 49 mm broad.

### DETAILED BOTANICAL DESCRIPTION

In mushroom breeding, mycelial (=vegetative) cultures of two compatible progenitors (typically these are haploid homokaryotic strains called homokaryons) must come into physical contact so that one or more fusion zones can occur between the progenitors. Within those fusion zones nuclei and organelles from the two progenitors become associated. In Agaricus bisporus, a novel, hybrid mycelium ultimately containing two compatible haploid nuclear types (one from each of the two progenitors) plus one mitochondrial type (from either one of the progenitors) emerges. This novel hybrid mycelium can be isolated and propagated to provide the new hybrid culture, which can be further subdivided and propagated for commercial or other purposes.

The hybrid strain of the present invention, ‘J10165’, is a cross between two parental heterokaryotic strains (each of which incorporates two compatible haploid nuclear types), ‘J9277’ and ‘B7023’. ‘J9277’ is the product of several generations of hybridization among diverse strains, including one tetrasporic wild progenitor as described in U.S. Pat. No. 5,304,721. ‘J9277’ is itself the subject of pending U.S. patent application Ser. No. 11/433,178, and its ancestry and construction, incorporating three wild strains and additional commercially cultivated strains, is detailed therein. A series of single-spore homokaryons (haploid offspring) was prepared from a spore print from ‘J9277,’ and one of these, called the ‘J9277-s45b’ homokaryon, was used in the ‘J10165’ cross.

The second homokaryon in the cross that produced ‘J10165’ is ‘B7023-s7’. ‘B7023’ is itself a hybrid strain owned by the Assignee of record, produced by the cross of wild homokaryon ‘I3-s13,’ isolated from a collection (‘I3’) from Israel, and homokaryon ‘S130-b,’ isolated from Sylvan's commercial hybrid strain ‘S-130’ in the ‘U1’ lineage group.

The cross was made by placing the homokaryons ‘J9277-s45b’ and ‘B7023-s7’ in close proximity on a sterile culture medium, allowing the two cultures to grow and contact each other, anastomose, and establish the hybrid heterokaryotic strain ‘J10165,’ which was then clonally propagated.

The pedigree of ‘J10165’ was confirmed by the ITS1+2 DNA sequence fingerprint. The positional notation for the Agaricus bisporus ITS1+2 DNA segment is taken from M. Challen et al., 95(1): 61-73 (2003), incorporated herein by reference. The presence of both C and T bases at 5 positions (52, 150, 153, 522, 563) in the ITS1+2 segment of ‘J10165’ demonstrates the presence of two homologous chromosomes, one contributed by ‘J9277-s45b’ and the other by ‘B7023-s7,’ each one of which carries either a C (only) or a T (only) at each specified position.

The performance characteristics and appearance of mushrooms produced in commercial cultivation will vary, depending on the properties of cultivation materials (compost, e.g.) used, crop management techniques employed, and environmental conditions such as air velocity, humidity, and CO₂ level. Some general characteristics of ‘J10165’ are similar to those of other commercially successful white hybrid mushroom strains, for which the ‘A-15’ hybrid strain may serve as an example. The timing of harvest of ‘J10165” is within 36 hours of that of ‘A-15,’ either faster or slower depending on growing conditions, and the yield is also comparable within a narrow range that is affected by specific conditions. The size and the proportions of the mushrooms are not statistically different between the two strains; both produce medium to large mushrooms by commercial grading standards, with broadly rounded caps. All of these traits are demanded by a large segment of the commercial market for mushrooms, and these similarities between ‘J0165’ and other commercial strains are a deliberate result of a particular selection and breeding strategy.

‘J10165’ can be distinguished from existing commercial hybrid white mushroom strains by at least three characteristics. First, it has a smoother cap than ‘A-15’. The cap of ‘J10165’ will remain smooth and even exhibit a reflective luster where its cap curvature is greatest, under conditions in which ‘A-15’ will develop a rough, scaly, non-lustrous cap surface. These traits can be observed in the drawing.

Second, ‘J10165’ exhibits cultural incompatibility toward ‘A-15,’ eliminating or greatly reducing the possibility of anastomosis between ‘J10165’ and current commercially grown strains. Consequently, it is expected that this will also prevent or greatly reduce the transmission or exchange of cytoplasmic elements including pathogenic viruses. This incompatibility can be demonstrated by confronting the two strains in cultivation. In commercial cultivation, an inoculum of the desired strain (spawn) is introduced into a prepared compost substrate and colonization proceeds for about two weeks. At that time, a layer of casing mix or ‘soil’ is applied to the upper surface of the compost to stimulate and support the production of a mushroom crop. Normally, a second inoculum (“CI” or “CAC”) of the same strain is introduced into the casing mix in order to speed and regularize the development of the mushroom crop. If two inocula constituting an incompatible combination are successively introduced, one into compost and the other into casing, the resulting antagonistic response reduces the production of mushrooms and/or delays their appearance, particularly away from the edges of the growing trays. This incompatibility response is the norm when two non-identical strains are used in this way.

The following Table I presents the results of Experiment 08-595, in which ‘J10165’ and ‘A-15’ were cultivated using compatible and incompatible combinations of inocula. All of the typical incompatability responses were observed.

TABLE 1
Effects of combining inocula of ‘J10165’ and ‘A-15’ in compatible
(self + self) and incompatible (self +non-self) combinations.

| | | | | |
|---|---|---|---|---|
| Compost inoculum | J10165 | J10165 | A-15 | A-15 |
| Casing inoculum | J10165 | A-15 | J10165 | A-15 |
| Total yield (% average, in g) | 119% | 107% | 75% | 114% |
| Days until first harvest | 16-17 | 16-17 | 18 | 17 |
| First mushrooms only at | no | yes | yes | no |
| edges of trays? | | | | |

The observed incompatibility indicates that ‘J10165’ will be less susceptible to infection by any “intergroup” contact with spores or mycelium of strains in the ‘U1’ lineage group, relative to the susceptibility of current commercial white hybrids strains in “intragroup” contacts with the same or other strains that are members of the commercially predominant ‘U1’ lineage group.

Third, ‘J10165’ incorporates a unique ITS DNA sequence fingerprint that distinguishes it from other known white hybrid mushrooms, namely, the presence of both C and T bases at positions 52, 150, 153, 522, and 563, both A and G bases at position 32, and a T base at position 461 in the ITS1+2 DNA segment of ‘J10165’. This fingerprint is unique with respect to those of the ‘U1’ and ‘U3’ lineage groups and also to those of hybrid strains ‘B7970’ and ‘J9277’ noted above. This fingerprint permits the identification of ‘J10165’ in both vegetative culture (e.g. inoculum, spawn, CI and CAC) and also in the mushroom stage as well. The uniqueness of this DNA segment is also indicative of the genetic novelty expected to characterize the entire genome. This supports the belief that other valuable novel characters may become evident as more experience is gained.

The ‘J10165’ strain provides for the abundant production of mushrooms having smooth, white caps. As The Royal Horticultural Society (R.H.S.) color charts do not provide a reference standard for the color “white”, direct measurements of color of the ‘J10165’ mushroom cap have been made using a Minolta Chromometer and the L-a-b color space system. Four measurements were made on the caps of each of seven mushrooms grown in a testing facility. The mean values, plus or minus the standard error, for the measured L, a, and b color components were as follows: L=91.7±0.19; a=−0.44±0.07; b=10.8±0.21. Colors within or substantially coinciding with the color space described by these three parameter distributions are called “white” according to standard and accepted practices of the commercial mushroom industry.

A formal description of the mushrooms produced by strain ‘J10165’ follows: Basidiomata agaricoid. Pileus at harvest stage broadly convex, 30-75 mm broad, surface white, glabrous and often lustrous. Flesh firm, white, typically 12-16 mm thick. Lamellae free, close, initially pallid, becoming dark chocolate brown, about RHS 187A-RHS 200A, as maturation progresses. Veil forming a thick, relatively inelastic, intermediate (semi-band-like with a wedge-shaped cross section) white annulus, smooth or obscurely striate above, smooth or floccose below. Stipe white, smooth, equal or slightly enlarged at base, 16-21 mm broad, length variable in response to cultural influences but often ca. 2.5 times the stipe thickness, interior stuffed-hollow. All parts generally do not develop pronounced non-white colors when rubbed, crushed or cut. Chemical reactions: KOH negative (not yellowing), Schaffer's Reaction (aniline×HNO₃) negative (neither yellow, red nor orange). Microscopic features are as previously described for the species in Kerrigan, R. W., The Agaricales of California. Vol. 6. Agaricaceae,Mad River Press, Arcata, Calif., 1986, the disclosure of which is incorporated herein by reference and as understood by those having ordinary skill in the art for the species Agaricus bisporus.

What is claimed is:

1. A new and distinct hybrid variety of Agaricus bisporus mushroom substantially as shown and described in the specification.
